(19) United States
(12) Patent Application Publication
(10) Pub. No.: US 2013/0152940 A1
(43) Pub. Date: Jun. 20, 2013

(54) Nasopharyngeal Trumpet with Inflatable Tip

(71) Applicants: IAN WALKER LARSON, HALF MOON BAY, CA (US); JEFFREY HOWARD LEVY, ALAMO, CA (US)

(72) Inventors: IAN WALKER LARSON, HALF MOON BAY, CA (US); JEFFREY HOWARD LEVY, ALAMO, CA (US)

(21) Appl. No.: 13/330,675

(22) Filed: Dec. 20, 2011

(51) Int. Cl. A61M 16/00

(52) U.S. Cl. 128/207.18

(57) ABSTRACT

An improved inflatable, tapered tip nasopharyngeal airway allows for comfortable self insertion and provides a clear airway for patient breathing in those with obstructive sleep apnea (OSA).

### BACKGROUND OF THE INVENTION

Anesthesiologists have long been using nasal trumpets to alleviate airway obstruction that commonly is seen in the surgically asleep patient. Part of any routine preoperative history and physical includes questions related to troubles in the past with sleeping, snoring, and or waking up tired. Many patients respond positively, and it is well known that the diagnosis of sleep apnea is expanding exponentially in the population as a whole. After an anesthetic induction, it was observed that even these drug induced sleep apnea patients breathed obstruction-free with a nasal trumpet. Studies have shown that anesthetic/drug induced sleep apnea models naturally occurring obstructive sleep apnea (OSA) The insertion of the current generation of nasal canula is uncomfortable and potentially traumatic. The inflatable tip of our device was designed to allow for easier and less traumatic insertion by a physician or during at home use by the patient. As the tip is collapsed and narrower and made of a softer material, it slides down the airway with ease and causes less trauma then standard nasal trumpet. A few of the issues with the current standard treatment for sleep apnea, the CPAP device, stem from the noise made from the motor providing a continuous flow of oxygen and the bulk of the mask which makes it uncomfortable for partners to sleep near the patients, and often causes a feeling of claustrophobia in the mask wearer.

### SUMMARY THE INVENTION

The invention herein is directed to a nasopharyngeal trumpet with an inflatable tip that maintains airway patency. The invention can be used to treat sleep apnea in the home by a patient placing into position before sleep.

The present invention is comprised of a semisoft malleable material such as polypropylene, PVC, polyurethane or polyethylene. The invention contains an inflatable tip which is inserted into the nostril until the trumpet is fully positioned with the distal end in the nasopharnx. The tip is then inflated via an inflation pouch to allow for airflow. The tip end of the present invention is inflatable ensuring ease of Insertion Into the nasal airway and is inflated through air delivery lines that traverse the entire length of the canula. The invention is sized for the patient.

### BRIEF DESCRIPTION OF THE DRAWINGS

FIG. 1 is a section view of the nasopharyngeal trumpet of the present invention in the inflated state in its embodiment.

FIG. 2 is an angled perspective view of the nasopharyngeal trumpet of the present invention with deflated tip.

FIG. 2a is a proximate end elevation view of the nasopharyngeal trumpet of the present invention.

FIG. 2b is a side view of the distal end in the nasopharyngeal trumpet of the present invention in the inflated state.

FIG. 2c is a cross section view taken along line A-A of the canula of FIG. 2 of preferred embodiment nasopharyngeal trumpet of the present invention.

FIG. 2d is a distal end elevation view of the nasopharyngeal trumpet of the present invention.

FIG. 3 is an angled perspective view of the trumpet demonstrating the alternate air delivery method of the present invention.

FIG. 3A is a cross section view demonstrating the single air inflation/deflation line of the present invention.

### DETAILED DESCRIPTION OF THE INVENTION

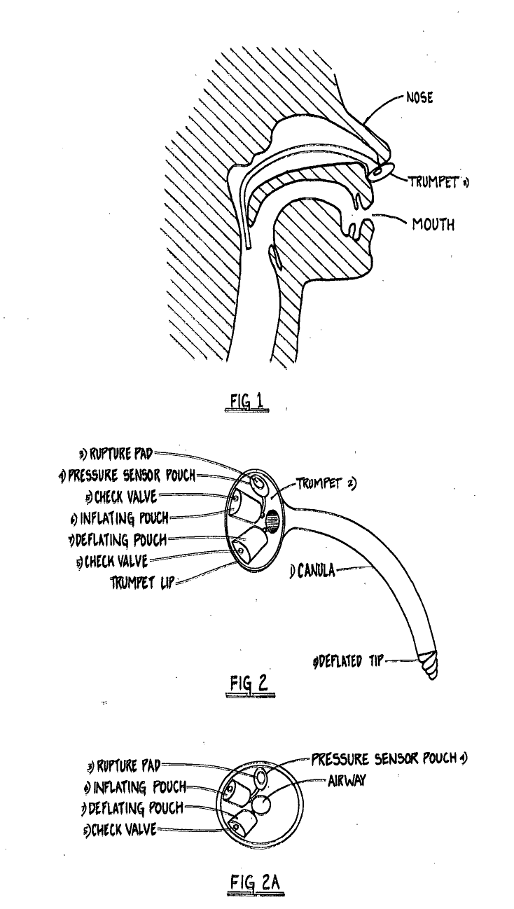

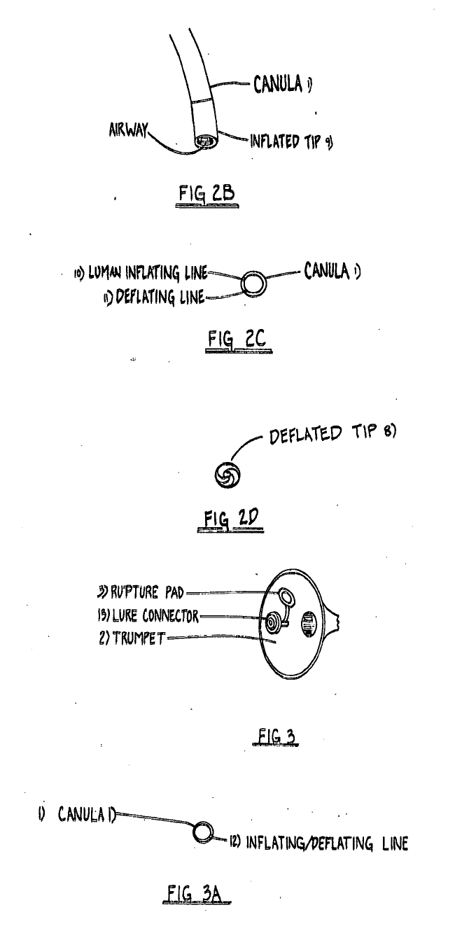

The tip end (FIG. 2.8 & 2b.9 & 2d.8) of the present invention is inflatable ensuring ease of insertion into the nasal airway when deflated. It is inflated through an air line (FIG. 2c.10-11) that traverses the entire length of the canula (FIG. 2.1, 2b.1, 2c.1). The tip end is compressed into a conical spiral like shape (FIGS. 2.8 & 2d.8) allowing an easy and consistent inflation once completely inserted. The trumpet end (FIGS. 2.2 & 2a.) is flared to ensure the device doesn't over insert nor get pulled into the patient's airway from breathing. The inflation mechanism (FIG. 2.6) for the tip is embedded into trumpet end of the device (FIG. 2a). It consists of a small pouch (FIG. 2a.6) that is pressed to allow air to travel through the shaft wall air line to the inflatable tip. A check valve (FIG. 2a.5) is included as the incoming regulator which keeps the pouch from deflating. The canula contains two air lines that are embedded into the canula, one for inflation air, one for deflation air (FIGS. 2c.10 & 2c.11). The inflation air line extends from the inflation pouch to the tip and the deflation air line extends from the tip to the deflation pouch. Alongside the inflating pouch is a pressure pad (FIGS. 2.4 & 2a.4) which allows the user to test the tip of inflating pressure. The pouch includes a small pressure regulator, or rupture pad (FIG. 2a.3), made up of thinner material which will burst prior to the tip being overinflating, protecting the nasal airway from damage in case of accidental over inflation and bursting. Next to the inflating pouch is a deflation pouch which consists of two check valves and a pouch (FIGS. 2a.5 & 2a.7). The check valves ensure that the airway doesn't deflate until intended. The inflatable tip is manufactured to be in a conical helix like shape without inflation (FIG. 2d) which inflates into a opened end. The inflatable tip is adhered to the canula wall via adhesive or welding. The device is manufactured in one shape that serves both nasal passages. An alternate air inflation/deflation method uses a standard medical lure connector (FIG. 3.13) where a patient connects a syringe to inflate and deflate the inflatable tip. In the alternate air delivery method a single air line (FIG. 3A.12) serves as the vehicle for both inflating and deflating the inflatable tip.

1. A nasopharyngeal airway trumpet comprising: a canula having a proximate end and a distal end and having a wall defining an airway passage extending from the proximate end to the distal end; air line means formed in said canula and extending the length of said canula; a tip attached to the distal end of the canula and communicating with said air line means, said tip being inflatable and deflatable through said air line means and when inflated forming an airway communicating with the airway passage of said canula.

2. The device of claim 1 wherein the distal end of said tip when deflated has a smaller diameter than said canula.

3. The device of claim 2 wherein when deflated the tip assumes a conical helix like shape.

4. The device of claim 1 wherein the canula means comprises two air lines formed in the wall of the canula, one of said air lines serving to inflate the tip and the other serving to deflate the tip.

5. The device of claim 1 wherein valve means are provided at the proximate end of the canula to control the inflation and deflation of said tip through said air line means.

6. The device of claim 4 wherein first and second valves are provided at the proximate end of the air lines, the first valve controlling the inflation of said tip through one of said air lines, and the second valve controlling the deflation of the tip through the other of said air lines.

7. The device of claim 6 wherein the valves are connected to the air lines through first and second pouches.

8. The device of claim 7 wherein the inflation pouch is provided with a pressure regulator.

9. The device of claim 8 wherein the pressure regulator comprises a rupture pad.

10. The device of claim 9 wherein the canula means comprises a single air line formed in the wall of the lumen serves to both inflate and deflate the tip.
